(19) United States
(12) Patent Application Publication
(10) Pub. No.: US 2026/0284429 A1
(43) Pub. Date: Sep. 24, 2026

(54) PHOTOTHERAPY DEVICE

(71) Applicant: InnoLux Corporation, Miao-Li County (TW)

(72) Inventors: Shun-Hsi WANG, Chu-Nan (TW); Chun-Kai LEE, Chu-Nan (TW); Fang-Iy WU, Chu-Nan (TW); Cheng-Hsu CHOU, Chu-Nan (TW)

(21) Appl. No.: 19/552,820

(22) Filed: Feb. 27, 2026

(30) Foreign Application Priority Data
Mar. 20, 2025 (CN) 202510331587.2

(51) Int. Cl. A61N 5/06

(52) U.S. Cl. CPC A61N 5/0625; A61N 2005/0632

(57) ABSTRACT

A phototherapy device is provided. The phototherapy device includes a substrate, a light-emitting unit, a driving structure and a first heat-conducting layer. The substrate includes a first surface, a second surface, and a plurality of through holes each connected between the first surface and the second surface. The light-emitting unit is disposed on the first surface. The driving structure is disposed in one of the plurality of through holes, and is electrically connected with the light-emitting unit. The heat-conducting layer is disposed in another one of the plurality of through holes.

### CROSS-REFERENCE TO RELATED APPLICATIONS

This application claims the benefits of the Chinese Patent Application Serial Number 202510331587.2, filed on Mar. 20, 2025, the subject matter of which is incorporated herein by reference.

### BACKGROUND

### Field of the Disclosure

The present disclosure relates to a phototherapy device and, more particularly, to the heat dissipation technology of the phototherapy device.

### Description of Related Art

To prevent excessively high surface temperature, the existing phototherapy device typically incorporates a heat dissipation design. For example, the design increase the area of the component's soldered electrodes or extend the heat-conducting area by laying large copper sheets or heat-dissipating patches, thereby improving the product's heat dissipation efficiency. However, this approach significantly reduces the product's flexibility (i.e., its bending resistance), making it difficult to attach the phototherapy device to the human body. Furthermore, to prevent short circuits due to exposed internal electrodes, an insulating protective layer is typically applied to protect the electrodes. However, the protective layer is often made of materials with low thermal conductivity, which may easily cause heat to accumulate in the product's internal circuit boards, resulting in negative effects such as increased thermal resistance, reduced component performance, increased product power consumption, and reduced heat dissipation efficiency.

Therefore, there is a need to provide an improved phototherapy device to alleviate and/or obviate the aforementioned problems.

### SUMMARY

The present disclosure provides a phototherapy device, which includes: a substrate including a first surface and a second surface opposite to each other, and a plurality of through holes connecting the first surface and the second surface; a light-emitting unit disposed on the first surface; a driving structure disposed in one of the plurality of through holes and electrically connected to the light-emitting unit; and a first heat-conducting layer disposed in another one of the plurality of through holes.

Other novel features of the disclosure will become more apparent from the following detailed description when taken in conjunction with the accompanying drawings.

### BRIEF DESCRIPTION OF DRAWINGS

FIG. 1A is a schematic diagram of a first surface of a phototherapy device according to an embodiment of the present disclosure;

FIG. 1B is a schematic diagram of a second surface of a phototherapy device according to an embodiment of the present disclosure;

FIG. 1C is a schematic diagram of a second surface of a phototherapy device according to another embodiment of the present disclosure;

FIG. 2 is a schematic diagram of the detailed structure of the phototherapy device according to an embodiment of the present disclosure;

FIG. 3 is a schematic diagram of the detailed structure of the phototherapy device according to another embodiment of the present disclosure;

FIG. 4A is a schematic diagram of first metal lines and second metal lines according to an embodiment of the present disclosure;

FIG. 4B is a schematic diagram of first metal lines and second metal lines according to another embodiment of the present disclosure; and

FIG. 5 is a schematic diagram of the second metal line and the driving structure according to an embodiment of the present disclosure.

### DETAILED DESCRIPTION OF EMBODIMENT

Reference will now be made in detail to exemplary embodiments of the present disclosure, examples of which are illustrated in the accompanying drawings. Wherever possible, the same reference numerals are used in the drawings and description to refer to the same or like parts.

Throughout the specification and the appended claims, certain terms may be used to refer to specific components. Those skilled in the art will understand that electronic device manufacturers may refer to the same components by different names. The present disclosure does not intend to distinguish between components that have the same function but have different names. In the following description and claims, words such as “containing” and “comprising” are open-ended words, and should be interpreted as meaning “including but not limited to”.

The terms, such as “about”, “substantially”, or “approximately” are generally interpreted as within 10% of a given value or range, or as within 5%, 3%, 2%, 1% or 0.5% of a given value or range.

In the specification and claims, unless otherwise specified, ordinal numbers, such as “first” and “second”, used herein are intended to distinguish components rather than disclose explicitly or implicitly that names of the components bear the wording of the ordinal numbers. The ordinal numbers do not imply what order a component and another component are in terms of space, time or steps of a manufacturing method. Thus, what is referred to as a “first component” in the specification may be referred to as a “second component” in the claims.

In the present disclosure, the terms “the given range is from the first numerical value to the second numerical value” and “the given range falls within the range from the first numerical value to the second numerical value” mean that the given range includes the first numerical value, the second numerical value, and other numerical values therebetween.

In addition, the device of the present disclosure may be used in electronic devices, and the electronic devices may include display devices, mechanical equipment, drug dispensing equipment, exposure devices, automotive devices, imaging devices, backlight devices, touch electronic devices, curved electronic devices or free shape electronic devices, but not limited thereto. The display device may, for example, include liquid crystal, light emitting diodes, fluorescence, phosphor, other suitable display media, or a combination thereof, but not limited thereto. The display device may be a non-self-luminous display device or a self-luminous display device. It should be noted that the electronic device may be any of the aforementioned arrangements and combinations, but not limited thereto. In addition, the electronic device may be a bendable or flexible electronic device. It should be noted that the electronic device may be any of the aforementioned arrangements and combinations, but not limited thereto. In addition, the appearance of the electronic device may be rectangular, circular, polygonal, a shape with curved edges, or other suitable shapes. The electronic device may have peripheral systems such as a driving system, a control system, a light source system, a shelf system, etc. to support the display device, the antenna device, or the tiled device. It is noted that the following are exemplary embodiments of the present disclosure, but the present disclosure is not limited thereto, while a feature of some embodiments can be applied to other embodiments through suitable modification, substitution, combination, or separation. In addition, the present disclosure can be combined with other known structures to form further embodiments.

Unless otherwise defined, all terms (including technical and scientific terms) used herein have the same meaning as commonly understood by those skilled in the art related to the present disclosure. It can be understood that these terms, such as those defined in commonly used dictionaries, should be interpreted as having meaning consistent with the relevant technology and the background or context of the present disclosure, and should not be interpreted in an idealized or excessively formal way, unless there is a special definition in the embodiment of the present disclosure.

In addition, the term “adjacent” in the specification and claims is used to describe mutual proximity, and does not necessarily mean mutual contact.

In addition, descriptions such as “when” or “while” in the present disclosure represent aspects such as “now, before or after”, and are not limited to situations that occur at the same time. In the present disclosure, similar descriptions such as “disposed on” refer to the corresponding positional relationship between the two components, and do not limit whether there is contact between the two components, unless otherwise specified. Furthermore, when the present disclosure provides multiple functions, if the word “or” is used between the functions, it means that the functions may exist independently, but it does not exclude that multiple functions may exist simultaneously.

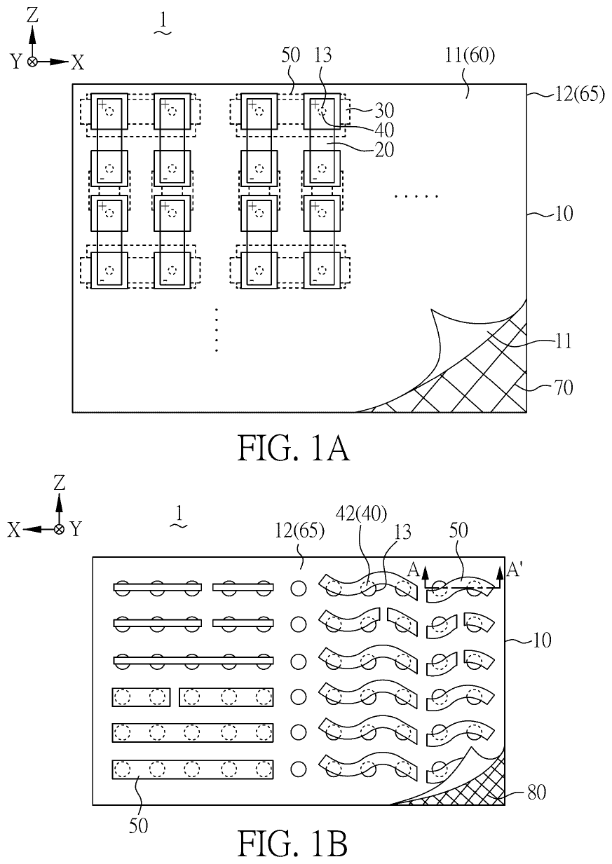

FIG. 1A is a schematic diagram of a first surface of a phototherapy device 1 according to an embodiment of the present disclosure. FIG. 1B is a schematic diagram of a second surface of a phototherapy device 1 according to an embodiment of the present disclosure. FIG. 1C is a schematic diagram of a second surface of a phototherapy device 1 according to another embodiment of the present disclosure. In some embodiments, the “first surface 11 of the phototherapy device 1” refers to the light-emitting surface of the light-emitting units 20, and the “second surface 12 of the phototherapy device 1” refers to the surface opposite the first surface in the Y direction. As shown in FIG. 1A, the phototherapy device 1 may include a substrate 10 and at least one light-emitting unit 20. For ease of explanation, a plurality of light-emitting units 20 will be taken as an example below. The substrate 10 may have a first surface 11 and a second surface 12, and the light-emitting unit 20 may be disposed on the first surface 11. In one embodiment, the light-emitting unit 20 may be, for example, a light-emitting diode, and the light-emitting unit 20 may have a P-polarity end (e.g., labeled “+”) and an N-polarity end (e.g., labeled “−”), but it is not limited thereto. Furthermore, a first insulating layer 60 may be disposed on the first surface 11 of the substrate 10. In one embodiment, the phototherapy device 1 may further include a first metal line 70, and in the Y direction, the first metal line 70 is disposed between the substrate 10 and the first insulating layer 60. The first metal line 70 may be arranged in a specific pattern, such as a grid, but it is not limited thereto. As shown in FIG. 1B and FIG. 1C, a second insulating layer 65 may be disposed on the second surface 12 of the substrate 10. In one embodiment, the phototherapy device 1 may further include a second metal line 80 disposed between the substrate 10 and the second insulating layer 65, and the second metal line 80 may be arranged in a specific pattern, such as a grid, but it is not limited thereto. In addition, in the Y direction, the substrate 10 may have a plurality of through holes 13 connecting the first surface 11 and the second surface 12. A first heat-conducting layer 40 may be disposed in a portion of the through holes 13 among the plurality of through holes 13, and the portion of the through holes 13 may penetrate to the second surface 12 of the substrate 10 and the second insulating layer 65, thereby forming a plurality of heat-conducting electrodes 42 on the second surface 12 of the substrate 10 and the second insulating layer 65. Furthermore, as shown in FIG. 1B, in some embodiments, the phototherapy device 1 further includes a second heat-conducting layer 50 disposed on the second surface 12. The first heat-conducting layer 40 and the heat-conducting electrodes 42 formed by the through holes 13 may be connected together via the second heat-conducting layer 50, but it is not limited thereto. As shown in FIG. 1C, in some embodiments, the heat-conducting electrodes 42 formed by the first heat-conducting layer 40 may not be connected together, and thus the phototherapy device 1 may not include the second heat-conducting layer 50.

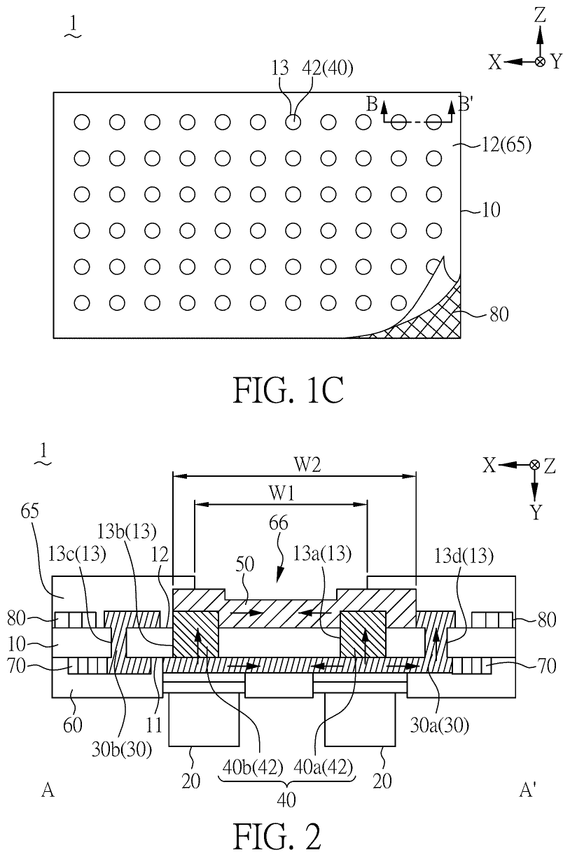

Next, the detailed structure of the phototherapy device 1 will be described. FIG. 2 is a schematic diagram of the detailed structure of the phototherapy device 1 according to an embodiment of the present disclosure, which is used to show the cross section taken along line A-A′ in FIG. 1B.

As shown in FIG. 2, the phototherapy device 1 may include a substrate 10, a light-emitting unit 20 disposed on the first surface 11 of the substrate 10, at least one driving structure 30, and the first heat-conducting layer 40. The substrate 10 includes a plurality of through holes 13. The at least one driving structure 30 may be disposed (for example, at least partially disposed) in one of the plurality of through holes 13. For example, a portion of the driving structure 30 may be disposed in one of the through holes 13, another portion of the driving structure 30 may be disposed on the first surface 11, and yet another portion of the driving structure 30 may be disposed on the second surface 12, while it is not limited thereto. The driving structure 30 is electrically connected to the light-emitting unit 20, and the first heat-conducting layer 40 may be disposed in another one of the plurality of through holes 13. In one embodiment, different from the through hole 13 in which the first heat-conducting layer 40 is disposed, the through hole 13 in which the driving structure 30 is disposed does not penetrate the first insulating layer 60 or the second insulating layer 65, but it is not limited thereto. In one embodiment, the driving structure 30 may serve as a driving trace of the light-emitting unit 20 for transmitting driving signals related to the light-emitting unit 20, but it is not limited thereto. In one embodiment, the phototherapy device 1 may have a plurality of driving structures 30, wherein a portion of the driving structures 30 may be electrically connected to one end (e.g., P-polarity end/N-polarity end) of the light-emitting unit 20, and the portion of the driving structures 30 is not electrically connected to the other end (e.g., N-polarity end/P-polarity end) of the light-emitting unit 20. Conversely, another portion of the driving structures 30 may be electrically connected to the other end (e.g., N-polarity end/P-polarity end) of the light-emitting unit 20, and the portion of the driving structures 30 is not electrically connected to the one end (e.g., P-polarity end/N-polarity end) of the light-emitting unit 20. Another one of the plurality of through holes 13 may be provided with a first heat-conducting layer 40, so that a heat-conducting electrode 42 may be formed on the substrate 10 by the first heat-conducting layer 40 in the through hole 13, thereby improving heat dissipation efficiency. It should be noted that the number of components described above is for example only and is not intended to be limiting.

In one embodiment, the phototherapy device 1 may further include a second heat-conducting layer 50, a first insulating layer 60, a second insulating layer 65, a first metal line 70, and/or a second metal line 80, but it is not limited thereto. In one embodiment, the first metal line 70 and the second metal line 80 are opposite to each other in the Y direction. For example, when the first metal line 70 is disposed on the first surface 11, the second metal line 80 may be disposed on the second surface 12. Furthermore, in the Y direction (e.g., in the normal direction of the substrate 10), the second heat-conducting layer 50 may overlap with at least a portion of the plurality of through holes 13.

In one embodiment, the second heat-conducting layer 50 may be connected to the first heat-conducting layer 40. For example, when the phototherapy device 1 includes a plurality of first heat-conducting layers 40 disposed in the plurality of through holes 13, each of the plurality of first heat-conducting layers 40 may be connected to a second heat-conducting layer 50. The plurality of first heat-conducting layers 40 may form a plurality of heat-conducting electrodes 42 on the substrate 10 (e.g., on the second surface 12). These heat-conducting electrodes 42 formed by the first heat-conducting layers 40 may be connected together by the plurality of second heat-conducting layers 50, for example, in a straight line or a serpentine manner, but it is not limited thereto. In one embodiment, the heat-conducting electrodes 42 formed by the first heat-conducting layers 40 of the same polarity are connected together by the second heat-conducting layer 50. Here, “same polarity” means that these heat-conducting electrodes 42 are electrically connected to one end of the light-emitting unit 20 (e.g., P polarity end/N polarity end) and are not electrically connected to the other end of the light-emitting unit 20 (e.g., N polarity end/P polarity end). That is, a plurality of heat-conducting electrodes 42 corresponding to the same polarity may be connected in series, thereby further improving the heat dissipation efficiency of the phototherapy device 1, while it is not limited thereto. Furthermore, by adjusting the width or size of the second heat-conducting layer 50, the heat dissipation efficiency of the phototherapy device 1 may also be adjusted. For example, the wider the width of the second heat-conducting layer 50, the higher the heat dissipation efficiency of the phototherapy device 1 may be, while it is not limited thereto.

In one embodiment, in the Y direction, the second insulating layer 65 may be disposed on the second heat-conducting layer 50, for example, covering at least a portion of the second heat-conducting layer 50. Furthermore, the second insulating layer 65 may also be disposed on the portion of the driving structure 30 disposed on the second surface 12 and/or the second metal line 80, for example, covering at least a portion of the driving structure 30 and at least a portion of the second metal line 80, while it is not limited thereto. In one embodiment, the second insulating layer 65 disposed on the second heat-conducting layer 50 may have an opening 66, which may expose at least a portion of the second heat-conducting layer 50. Thus, at least a portion of the heat energy conducted by the second heat-conducting layer 50 may be conducted to the air, thereby significantly improving the heat dissipation efficiency of the phototherapy device 1, while it is not limited thereto.

In one embodiment, the opening 66 may have a width W1 in the X-direction. In some embodiments, the width W1 may refer to the bottom width of the opening 66 in the X-direction. In other embodiments, the width W1 may refer to the maximum width of the opening 66 in the X-direction. The second heat-conducting layer 50 may have a width W2 in the X-direction. The width W1 of the opening 66 may be smaller than the width W2 of the second heat-conducting layer 50 (W1<W2), but it is not limited thereto), which can enhance the flexural reliability of the phototherapy device 1.

In one embodiment, the area of the opening 66 may affect the heat dissipation efficiency. Referring to FIG. 1B, in one embodiment, the area of the opening 66 may be, for example, the area on the XY plane. In one embodiment, a plurality of light-emitting units 20 may be provided on the first surface 11, and a plurality of second heat-conducting layers 50 and a plurality of openings 66 may be provided on the second surface 12. In one embodiment, in a top view, the plurality of light-emitting units 20 may have a surrounding area, such as the total area occupied by the plurality of light-emitting units 20 on the first surface 11, or the area enclosed by the boundary line formed by connecting the outermost plurality of light-emitting units 20 on the first surface 11, while it is not limited thereto. In a top view, the area of the opening 66 is the area of the opening 66 on the XY plane. Furthermore, the total area of the plurality of openings 66 and the surrounding area of the plurality of light-emitting units 20 may have a ratio. In one embodiment, the ratio may be between 3% and 50% (3%≤ratio≤50%), but it is not limited thereto. In another embodiment, the ratio may be between 5% and 30% (5%≤the ratio≤30%), so that the phototherapy device 1 has better heat dissipation and reliability, but it is not limited thereto.

In one embodiment, in the Y direction, the first insulating layer 60 may be arranged on the portion of the driving structure 30 disposed on the first surface 11 and the first metal line 70, for example, it may cover at least a portion of the driving structure 30 and at least a portion of the first metal line 70. In addition, the first insulating layer 60 may be disposed between the light-emitting unit 20 and the substrate 10, while it is not limited thereto.

In one embodiment, the first metal layer 70 and the second metal layer 80 partially may not overlap in the Y direction, for example, the first metal layer 70 and the second metal layer 80 may not completely overlap, which will be described in more detail in the subsequent paragraphs. In one embodiment, the first metal layer 70 may be disposed on the same layer as the driving structure 30, and/or the second metal layer 80 may be disposed on the same layer as the driving structure 30, and the first metal layer 70 and the second metal layer 80 are disposed in an area on the substrate 10 that does not have the driving structure 30 or other components, such as a dummy region, but it is not limited thereto. The first metal layer 70 and the second metal layer 80 may be used to enhance the flexibility of the phototherapy device 1, which will be described in more detail in the subsequent paragraphs.

Next, the materials of the various components will be described. In one embodiment, substrate 10 may be, for example, a flexible printed circuit (FPC) substrate or a bendable circuit substrate, and its material may include, for example, polyimide (PI), polyethylene terephthalate (PET), or other suitable soft materials, or a combination thereof, while it is not limited thereto. In one embodiment, the substrate 10 may have a single-layer, double-layer, or multi-layer structure.

In one embodiment, the material of the driving structure 30 may include various suitable conductive materials.

In one embodiment, the material of the first heat-conducting layer 40 may include a high thermal conductivity material, such as a metal material including copper, silver paste, etc., but is not limited to metal. Any material with high thermal conductivity may be implemented, while is not limited thereto.

In one embodiment, the material of the second heat-conducting layer 50 may include a material with a high thermal conductivity coefficient, such as a metal material including copper or silver paste, but is not limited to metals. Any material with high thermal conductivity may be used, while it is not limited thereto. In addition, in one embodiment, the surface of the second heat-conducting layer 50 exposed to the air through the opening 66 and/or the surface of the first heat-conducting layer 40 exposed to the through hole 13 may be covered with a high thermal conductivity and oxidation-resistant material layer (not shown in the figure). The material of the material layer may include, for example, nickel, gold, or other suitable materials, or a combination thereof, while it is not limited thereto, so as to enhance the oxidation resistance and increase the reliability of the product. In addition, the surface of the second heat-conducting layer 50 exposed to the air through the opening 66 and/or the surface of the first heat-conducting layer 40 exposed to the through hole 13 may also be further provided with thermal paste or a heat sink (not shown in the figure) to dissipate heat energy more quickly, while it is not limited thereto.

In one embodiment, the first metal line 70 and/or the second metal line 80 may be made of a high thermal conductivity material, such as copper, silver paste, metal nanoparticles or nanowires, graphene or other suitable materials, or a combination thereof, but it is not limited thereto.

The detailed connection methods and variations between the components are described below. In the example of FIG. 2, the through hole 13 may include through holes 13a~13d, the driving structure 30 may include a driving structure 30a disposed in the through hole 13d and a driving structure 30b disposed in the through hole 13c, and the first heat-conducting layer 40 may include a first heat-conducting layer 40a disposed in the through hole 13a and a first heat-conducting layer 40b disposed in the through hole 13b. In the X direction, the driving structure 30a and the driving structure 30b are disposed opposite each other in the X direction, for example, and correspond to different polarities (for example, respectively connected to different polarity ends of the light-emitting unit 20), and the first heat-conducting layer 40a and the first heat-conducting layer 40b are disposed opposite to each other in the X direction, for example, but are not limited thereto. The second heat-conducting layer 50 connects the first heat-conducting layer 40a and the first heat-conducting layer 40b. The second heat-conducting layer 50 may be electrically connected to one of the driving structure 30a and the driving structure 30b, and not electrically connected to the other one. For example, in the example of FIG. 2, the second heat-conducting layer 50 is electrically connected to the driving structure 30a, but not electrically connected to the driving structure 30b.

In a state where the second heat-conductive layer 50 is electrically connected to one of the driving structure 30a and the driving structure 30b, furthermore, in one embodiment, the first metal line 70 may be electrically connected to one of the driving structure 30a and the driving structure 30b, the first metal line 70 may be not electrically connected to the other one of the driving structure 30a and the driving structure 30b, and the second metal line 80 may be not electrically connected to both the driving structure 30a and the driving structure 30b, wherein the first metal line 70 and the one of the driving structure 30a and the driving structure 30b correspond to the same polarity (that is, both are connected to the same polarity end of the light-emitting unit 20). For example, in the example of FIG. 2, the first metal line 70 is electrically connected to the driving structure 30a corresponding to the same polarity and is not electrically connected to the driving structure 30b of a different polarity, and the second metal line 80 is not electrically connected to both the driving structure 30a and the driving structure 30b. In another embodiment, the second metal line 80 may be electrically connected to one of the driving structure 30a and the driving structure 30b and corresponds to the same polarity, while the second metal line 80 is not electrically connected to the other one of the driving structure 30a and the driving structure 30b, and the first metal line 70 is not electrically connected to either of the driving structure 30a and the driving structure 30b. Furthermore, in yet another embodiment, the first metal line 70 and the second metal line 80 are both electrically connected to one of the driving structure 30a and the driving structure 30b and correspond to the same polarity, while the first metal line 70 and the second metal line 80 are not electrically connected to the other one of the driving structure 30a and the driving structure 30b. However, the present disclosure is not limited thereto.

Furthermore, different from the aforementioned example, the second heat-conducting layer 50 may be not electrically connected to both the driving structure 30a and the driving structure 30b. In a state that the second heat-conducting layer 50 is not electrically connected to both the driving structure 30a and the driving structure 30b, furthermore, in one embodiment, the first metal line 70 is electrically connected to one of the driving structure 30a and the driving structure 30b and corresponds to the same polarity, the first metal line 70 is not electrically connected to the other one of the driving structure 30a and the driving structure 30b, and the second metal line 80 is not electrically connected to both the driving structure 30a and the driving structure 30b. Furthermore, in another embodiment, the second metal line 80 is electrically connected to one of the driving structure 30a and the driving structure 30b and corresponds to the same polarity, the second metal line 80 is not electrically connected to the other one of the driving structure 30a and the driving structure 30b, and the first metal line 70 is not electrically connected to both the driving structure 30a and the driving structure 30b. In another embodiment, the first metal line 70 and the second metal line 80 are both electrically connected to one of the driving structure 30a and the driving structure 30b and correspond to the same polarity, and the first metal line 70 and the second metal line 80 are not electrically connected to the other one of the driving structure 30a and the driving structure 30b. However, the present disclosure is not limited thereto.

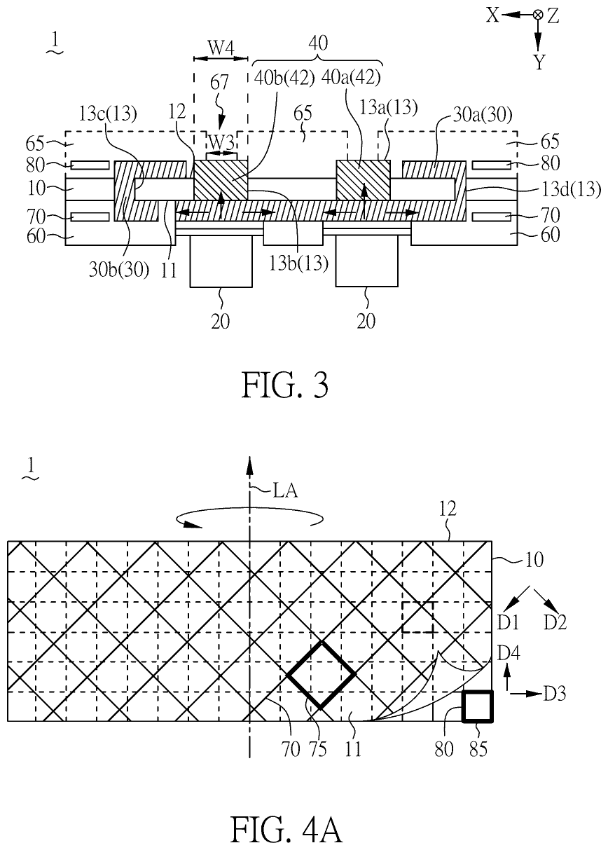

The phototherapy device 1 of the present disclosure may also have different structural designs. FIG. 3 is a schematic diagram of the detailed structure of the phototherapy device 1 according to another embodiment of the present disclosure, which is used to illustrate other aspects of the cross-section taken along line B-B′ in FIG. 1C, and please refer to FIG. 1A to FIG. 2 for reference.

In the example of FIG. 3, the phototherapy device 1 may not include the second heat-conducting layer 50. In this configuration, the second insulating layer 65 may cover a portion of the through hole 13. For example, the second insulating layer 65 may have a second opening 67 corresponding to the position of the first heat-conducting layer 40. The second opening 67 may correspond to the position of the through hole 13. Therefore, at least a portion of the first heat-conducting layer 40 may be exposed to the second opening 67 and exposed to the air. In one embodiment, the second opening 67 may have a width W3 in the X-direction, and the first heat-conducting layer 40 may have a width W4 in the X-direction, wherein the width W3 of the second opening 67 is smaller than the width W4 of the first heat-conducting layer 40 (W3<W4), so as to increase the reliability of the phototherapy device 1. Thus, another structural aspect of the phototherapy device 1 can be understood.

In addition, the phototherapy device 1 of the present disclosure may have a bendable structure. For example, the phototherapy device 1 may be bent in one direction. In some embodiments, the phototherapy device 1 may be bent along a bending axis LA (shown in FIG. 4A) so that the regions of the phototherapy device 1 on both sides of the bending axis LA may have an angle (not 180 degrees). In this way, the regions of the phototherapy device 1 on both sides of the bending axis LA may have different normal directions, and thus are suitable for being attached to non-planar surfaces, such as human body parts, but it is not limited thereto. In order to enhance the flexibility of the phototherapy device 1, the phototherapy device 1 may have specially designed first metal line 70 and second metal line 80.

FIG. 4A is a schematic diagram of first metal lines 70 and second metal lines 80 according to an embodiment of the present disclosure, and please refer to FIG. 1A to FIG. 3 for reference. It should be noted that, to clearly illustrate the structure of the first metal line 70 and the second metal line 80, FIG. 4A only shows the first metal lines 70 and the second metal lines 80 disposed on the substrate 10, while hiding other components. Furthermore, the metal lines disposed on the first surface of the substrate 10 are shown as solid lines, while the metal lines disposed on the second surface are shown as dashed lines.

As shown in FIG. 4A, a plurality of first metal lines 70 may be disposed on the first surface 11 of the substrate 10, and the plurality of first metal lines 70 may be staggered to form a plurality of first grid structures 75. In one embodiment, the first grid structure 75 may be, for example, a rectangle, triangle, trapezoid, rhombus, or polygon, or may be a repeating grid structure of other irregular shapes, but it is not limited thereto. In one embodiment, the plurality of first grid structures 75 may have the same size or different sizes, and may have different proportions or shapes. Furthermore, the plurality of first metal lines 70 may have at least two extension directions. For example, a portion of the plurality of first metal lines 70 may have a first extension direction D1, another portion of the plurality of first metal lines 70 may have a second extension direction D2, and the first extension direction D1 and the second extension direction D2 may be different, but it is not limited thereto. It should be noted that, although the plurality of first metal lines 70 have two extension directions D1 and D2 in the example of FIG. 4A, the plurality of first metal lines 70 may also have three or more extension directions in other embodiments, while it is not limited thereto. In one embodiment, the phototherapy device 1 is of a bendable structure and has a bending axis LA. For example, the phototherapy device 1 itself may be bent along the bending axis LA, while it is not limited thereto. In one embodiment, the first extension direction D1 of the first metal line 70 is different from the bending axis LA, that is, there is an angle therebetween. In one embodiment, the second extension direction D2 of the first metal line 70 is different from the bending axis LA, that is, there is an angle therebetween. In one embodiment, both the first extension direction D1 and the second extension direction D2 of the first metal line 70 are different from the bending axis LA. In one embodiment, different from the example of FIG. 4A, when the first metal lines 70 have three or more extension directions, each extension direction may be different from the bending axis LA, while it is not limited thereto.

In addition, a plurality of second metal lines 80 may be disposed on the second surface 12 of the substrate 10, and the plurality of second metal lines 80 may be arranged in a staggered manner to form a plurality of second grid structures 85. In one embodiment, the second grid structure 85 may be, for example, a rectangle, triangle, trapezoid, rhombus, or polygon, or may be a repeating grid structure of other irregular shapes, but it is not limited thereto. In one embodiment, the plurality of second grid structures 85 may have the same size or different sizes, and may also have different proportions or shapes. In addition, the plurality of second metal lines 80 have at least two extension directions. For example, a portion of the plurality of second metal lines 80 may have an extension direction D2, another portion of the plurality of second metal lines 80 may have another extension direction D4, and the extension direction D2 is different from the extension direction D4, but it is not limited thereto. It should be noted that, although the plurality of second metal lines 80 have two extension directions D3 and D4 in the example of FIG. 4A, the plurality of second metal lines 80 may have more than three extension directions in other embodiments, while it is not limited thereto. Furthermore, in one embodiment, the first metal lines 70 and the second metal lines 80 are opposed to each other in the Y direction, and the first metal lines 70 and the second metal lines 80 at least partially do not overlap. For example, in the Y direction, each first metal line 70 does not completely overlap with the plurality of second metal lines 80, while it is not limited thereto. Furthermore, in one embodiment, the shape of the first grid structure 75 formed by the first metal lines 70 may differ from the shape or size of the second grid structure 85 formed by the second metal lines 80. For example, each first grid structure 75 does not completely overlap with the second grid structure 85 when moved in either the X or Z direction, while it is not limited thereto. Furthermore, the first metal line 70 itself des not completely overlap with the second metal line 80 itself.

In the example of FIG. 4A, since the first extension direction D1 and the second extension direction D2 of the first metal line 70 are different from the bending axis LA, the provision of the first metal line 70 may enhance the flexibility of the phototherapy device 1, but it is not limited thereto. Furthermore, the flexibility of the phototherapy device 1 may also be enhanced by the design of the first metal line 70 and the second metal line 80 that do not completely overlap, but it is not limited thereto.

It should be noted that, although FIG. 4A illustrates the example in which the first extension direction D1 and the second extension direction D2 of the first metal line 70 are different from the bending axis LA, the third extension direction D3 and the fourth extension direction D4 of the second metal line 80 may also be different from the bending axis LA. Alternatively, the extension direction of the first metal line 70 and the extension direction of the second metal line 80 may both be different from the bending axis LA, while it is not limited thereto. In other words, the extension direction of at least one of the first metal line 70 and the second metal line 80 of the present disclosure is different from the bending axis LA, thereby enhancing the flexibility of the phototherapy device 1.

FIG. 4B is a schematic diagram of first metal lines 70 and second metal lines 80 according to another embodiment of the present disclosure, and please refer to FIG. 1A to FIG. 4A for reference. The example in FIG. 4B is generally applicable to the description of the example in FIG. 4A, and thus the following description mainly focuses on the differences.

As shown in FIG. 4B, a first patterned structure 76 may be provided within at least a portion of the first grid structures 75. In some embodiments, the first patterned structure 76 and the first metal line 70 may be fabricated simultaneously. In some embodiments, the first patterned structure 76 and the first metal line 70 may be formed in the same layer. The material of the first patterned structure 76 may be the same as that of the first metal line 70, but it is not limited thereto. Furthermore, a second patterned structure 86 may be provided within at least a portion of the second grid structures 85. In some embodiments, the second patterned structure 86 and the second metal line 80 may be fabricated simultaneously. In some embodiments, the second patterned structure 86 and the second metal line 80 may be formed in the same layer. The material of the second patterned structure 86 may be the same as that of the second metal line 80, but it is not limited thereto. In one embodiment, the first patterned structures 76 in different grids may have the same or different shapes, or may have the same shape but are scaled at different ratios. In one embodiment, the second patterned structures 86 in different grids may have the same or different shapes, or may have the same shape but are scaled at different ratios.

The features of the first metal line 70, the second metal line 80, the first grid structure 75 and the second grid structure 85 in the example of FIG. 4B are applicable to the various descriptions of the example of FIG. 4A, and these features are not affected by the first patterned structure 76 and the second patterned structure 86, so that a detailed description is deemed unnecessary.

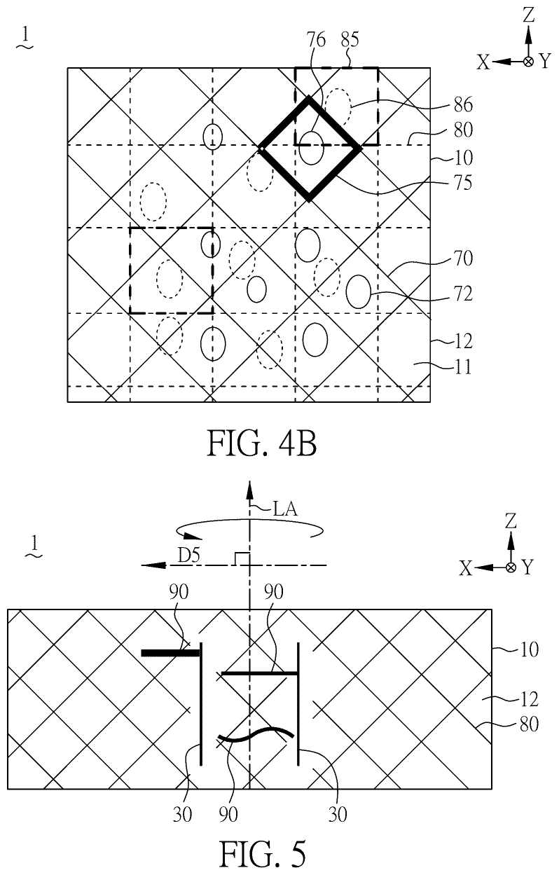

The present disclosure may also include various embodiments. FIG. 5 is a schematic diagram of the second metal line 80 and the driving structure 30 according to an embodiment of the present disclosure, and please refer to FIG. 1A to FIG. 4B for reference. It should be noted that, to clearly illustrate the structural features, FIG. 5 only shows the second metal line 80 and the driving structure 30 disposed on the second surface 12 of the substrate 10, and hides other components.

As shown in FIG. 5, a plurality of second metal lines 80 and a plurality of driving structures 30 (e.g., a plurality of driving signal lines) may be disposed on the second surface 12 of the substrate 10, wherein the second metal lines 80 may be disposed in an area outside the driving structures 30. The detailed features of the second metal lines 80 and the driving structures 30 are applicable to the description of the various embodiments in FIG. 1A to FIG. 4B, and thus a detailed description is deemed unnecessary. Furthermore, the phototherapy device 1 may further include at least one reinforcing line 90 disposed on the substrate 10 (e.g., disposed on the second surface 12 of the substrate 10) and having a fifth extension direction D5. In one embodiment, the fifth extension direction D5 may be perpendicular to the bending axis LA, but it is not limited thereto. In one embodiment, the reinforcing line 90 may be connected to at least a portion of the driving structure 30, but it is not limited thereto. In one embodiment, the material of the reinforcing line 90 may be metal, such as copper, but it is not limited thereto. In one embodiment, the shape of the reinforcing line 90 may be, for example, a straight line, a serpentine line, or a block, but it is not limited thereto. In this way, the reinforcing lines 90 may improve the folding resistance of the driving structure 30, thereby improving the flexibility of the phototherapy device 1, but it is not limited thereto. In one embodiment, each reinforcing line 90 may also be connected to other reinforcing lines 90, the first metal lines 70, and/or the second metal lines 80 connected to the driving structures 30 of the same polarity, but it is not limited thereto.

It should be noted that, although the example of FIG. 5 is described with the reinforcing line 90 being set on the second surface 12, the reinforcing line 90 may also be designed to be set on the first surface 11, or the reinforcing line 90 may be designed to be set on both the first surface 11 and the second surface 12, while it is not limited thereto.

Accordingly, the features of the phototherapy device 1 of the present disclosure can be understood.

In one embodiment, it may determine whether a product in contention falls within the protection scope of the present disclosure at least by the presence or absence of components, component configurations and/or physical appearance observation of the product in contention, or by the manufacturing method of the product in contention, while it is not limited thereto.

It can be seen that the phototherapy device 1 provided in the present disclosure has a specially designed through hole 13, first heat-conducting layer 40 and/or second heat-conducting layer 50, so as to facilitate the improvement of the heat dissipation effect of the phototherapy device 1. Alternatively, the phototherapy device 1 of the present disclosure has a special metal line design to improve the flexibility of the phototherapy device 1.

The details or features of the various embodiments of the present disclosure may be mixed and matched as long as they do not violate the spirit of the invention or conflict with each other.

The aforementioned specific embodiments should be construed as merely illustrative, and not limiting the rest of the present disclosure in any way.

1. A phototherapy device, comprising:
a substrate including a first surface and a second surface opposite to each other, and a plurality of through holes connecting the first surface and the second surface;
a light-emitting unit disposed on the first surface;
a driving structure disposed in one of the plurality of through holes and electrically connected to the light-emitting unit; and
a first heat-conducting layer disposed in another one of the plurality of through holes.

2. The phototherapy device as claimed in claim 1, further comprising a second heat-conducting layer disposed on the second surface and connected to the first heat-conducting layer.

3. The phototherapy device as claimed in claim 2, further comprising an insulating layer disposed on the second heat-conducting layer, wherein the insulating layer has an opening, and the opening exposes the second heat-conducting layer.

4. The phototherapy device as claimed in claim 2, wherein, in a normal direction of the substrate, the second heat-conducting layer overlaps at least a portion of the plurality of through holes.

5. The phototherapy device as claimed in claim 3, wherein, in a top view direction, the phototherapy device comprises a plurality of light-emitting units, a plurality of insulating layers and a plurality of openings, and a ratio of a total area of the plurality of openings to a surrounding area of the plurality of light-emitting units is between 3 percent and 50 percent.

6. The phototherapy device as claimed in claim 1, further comprising a first metal line disposed on the first surface or the second surface, wherein the first metal line has a first extension direction, the substrate has a bending axis, and the first extension direction is different from the bending axis.

7. The phototherapy device as claimed in claim 6, further comprising a second metal line disposed on the substrate and opposite to the first metal line, wherein the first metal line and the second metal line partially do not overlap.

8. The phototherapy device as claimed in claim 1, further comprising a reinforcing line disposed on the substrate, wherein an extension direction of the reinforcing line is perpendicular to a bending axis of the substrate.

9. The phototherapy device as claimed in claim 8, wherein the reinforcing line is connected to the driving structure.

10. The phototherapy device as claimed in claim 1, further comprising a plurality of second heat-conducting layers disposed on the second surface, wherein at least a portion of the plurality of second heat-conducting layers are connected to each other.

11. The phototherapy device as claimed in claim 3, wherein the another one of the plurality of through holes penetrates through the second surface and the insulating layer, and the first heat-conducting layer disposed in the another one of the plurality of through holes forms a heat-conducting electrode on the second surface and the insulating layer.

12. The phototherapy device as claimed in claim 11, wherein, in a top view direction, the phototherapy device comprises a plurality of heat-conducting electrodes, and the plurality of heat-conducting electrodes are connected together via the second heat-conducting layer.

13. The phototherapy device as claimed in claim 3, wherein, in a top view direction, the insulating layer covers at least a portion of the driving structures.

14. The phototherapy device as claimed in claim 3, wherein the opening has a first width, the second heat-conducting layer has a second width, and the first width is smaller than the second width.

15. The phototherapy device as claimed in claim 3, further comprising another insulating layer, which, in a top view direction, covers at least a portion of the driving structure and is disposed between the light-emitting unit and the substrate.

16. The phototherapy device as claimed in claim 1, further comprising an insulating layer, wherein the insulating layer covers a portion of the plurality of through holes.

17. The phototherapy device as claimed in claim 16, wherein the insulating layer has another opening corresponding to the first heat-conducting layer, the another opening corresponds to the another one of the plurality of through holes, and at least a portion of the first heat-conducting layer is exposed in the another opening.

18. The phototherapy device as claimed in claim 17, wherein the another opening has a third width, the first heat-conducting layer has a fourth width, and the third width is smaller than the fourth width.

19. The phototherapy device as claimed in claim 6, wherein the phototherapy device comprises a plurality of first metal lines arranged in a staggered manner to form a plurality of first grid structures, and a first patterned structure is disposed within at least one of the first grid structures.

20. The phototherapy device as claimed in claim 7, wherein the phototherapy device comprises a plurality of second metal lines arranged in a staggered manner to form a plurality of second grid structures, and a second patterned structure is disposed within at least one of the second grid structures.
